(19) United States
(12) Patent Application Publication
(10) Pub. No.: US 2007/0100315 A1
(43) Pub. Date: May 3, 2007

(54) Fluid-flow cassette for an ophthalmic surgical instrument

(76) Inventor: Samuel Dean Traxinger, Selma, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/583,302

(22) Filed: Oct. 18, 2006

Related U.S. Application Data
(62) Division of application No. 10/862,916, filed on Jun. 7, 2004.
(60) Provisional application No. 60/476,491, filed on Jun. 6, 2003.

(51) Int. Cl. A61M 5/00; A61M 31/00

(52) U.S. Cl. 604/500; 604/111

(57) ABSTRACT

A cartridge or cassette adapted to be attached to an apparatus for controlling the flow of fluid to and from a surgical handpiece. The cassette includes irrigation and aspiration fluid-flow paths and consisting of interconnected tubing having inlet and outlet port's associated with the case of the cassette that are accessible for attachment to fluid and vacuum sources. The rear cover of the case includes cutouts enabling the cassette to be releasably and registerably attached to a surgical instrument. A sensor on the instrument engages a portion of the cassette to verify that the cassette is correctly positioned and is appropriate for use with the instrument. Locking means on the instrument releasably engage the cassette in position. Irrigation fluid may be redirected into the aspiration channel of the handpiece in response to a reflux signal.

### CROSS REFERENCE TO RELATED APPLICATION

This is a divisional application of U.S. Ser. No. 10/862,916, which was filed on Jun. 7, 2004.

### FIELD OF THE INVENTION

A fluid-flow cassette adapted to be attached to a surgical instrument for controlling the flow of fluids to and from a surgical handpiece.

### BACKGROUND

In the treatment of cataracts, a phacoemulsification instrument is widely used for the fragmentation and removal of a crystalline lens before replacing the defective lens with an artificial lens. Such an apparatus requires the administration of a preferred level of ultrasonic energy to the lens, the introduction of an irrigation fluid stream to the operative site, and the vacuum removal of fragmented tissue from the operative site via an aspiration channel. Control signals are supplied to the instrument usually by means of a foot operable controller. The foot operable controller includes independent means for generating control signals as required for establishing the desired ultrasonic power level and irrigation fluid flow and for varying the aspiration vacuum pump in response to foot movement.

In one prior approach, a fluid-flow control apparatus adapted for use with an ultrasonic surgical tool that provides for irrigation of a surgical site and for aspirating fluid from the surgical site comprised several other components. These components include a source of irrigation fluid, an irrigation fluid conduit for conducting the irrigation fluid to a surgical site, an aspiration fluid conduit for conducting fluid away from the surgical site, a suction pump connected to the aspiration fluid conduit for aspirating fluid from the surgical site, a pressure-sensitive control system for removing the source of suction from the aspiration conduit when a predetermined value of suction is exceeded, and a valve for controllably admitting irrigation fluid into the aspiration fluid conduit. A check valve in the irrigation conduit prevented a reverse surge when the irrigation fluid was admitted to the aspiration conduit.

In another prior approach, a phacoemulsification apparatus operable for maintaining the proper pressure in the surgical site within the eye when blockage occurs in the aspiration conduit of the phacoemulsifier surgical handpiece has been used. A blockage or occlusion may occur, for example, when a piece of fragmented tissue which is larger than the axial bore of the aspiration conduit within the needle is drawn against the entrance to the axial bore in the needle. When such a blockage occurs in the aspiration line, the negative pressure or suction in the aspiration conduit between the surgical site and the vacuum pump increases. If the blockage is then suddenly released either by the mechanical action of the ultrasonic tool or by the increased value of the suction force, there is a tendency for the fluid within the surgical site to suddenly rush into the aspiration conduit with perhaps disastrous consequences. The flow control system of this approach provided for automatic rapid equalization of the pressure in the aspiration conduit when the occlusion was removed. This was accomplished by providing a flow-sensitive transducer in the aspiration conduit that sensed the rate of fluid flow and generated an electrical signal that was sent to a controller. Whenever the flow rate increased suddenly, indicating that a blockage has just been cleared, the controller caused a vent valve in the aspiration conduit to open at once, thus relieving the suction and preventing excessive withdrawal of fluid from the surgical site.

In another variation of a method for controlling irrigation and aspiration of fluids from the eye, irrigation fluid was supplied to an enclosed surgical site such as the interior of the eye and withdrawn from the surgical site through an aspiration conduit. A flow-sensitive transducer in the aspiration conduit sensed the sudden increase in flow that occurs when a blockage in the aspiration tube is released, and actuated a valve that released fluid from a second fluid source into the aspiration line. At the same time, the aspiration pump is shut off until the flow rate had returned approximately to normal. In this way, the surge of fluid out of the eye when an aspiration line blockage was released was greatly diminished.

In yet another prior approach, a phacoemulsification apparatus was used wherein irrigation fluid was supplied to the surgical site from a source of fluid via an irrigation conduit provided with a pressure relief valve to prevent the irrigation pressure from becoming too high. Similarly, the aspiration conduit was provided with a relief vent valve that opened to the atmosphere at a preset pressure differential, thereby preventing the suction in the aspiration conduit from exceeding a preset value. In this way, the suction in the aspiration line never exceeded a predetermined preset value, and the surgical site was not exposed to excess suction when a blockage was cleared.

Many instruments used in surgical procedures such as phacoemulsification utilize disposable units such as cassettes for controlling the flow of fluids. Experiences with contamination of reused units and components, together with the availability of low cost, but high performance, moldable materials, have stimulated this trend. Consequently, where a sterile fluid or material is to be passed to a surgical site, or various sterile flows are to be combined, systems are designed so that each surgical procedure is carried out using a new tubing set. Further benefits are derived with these systems from the fact that peristaltic pumps, using movable rollers, can operate directly on the tubing exterior to advance the fluid, or conversely, to block flow in a tubing simply by externally pinching the tubing. Neither event affects the sterility of the internal tubing in ordinary operation.

In typical practice, pre-sterilized disposable tubing sets are configured for particular applications, sometimes including small disposable accessories as well. Before use, the operator removes the tubing set from a sterile container or package and, under sterile conditions, makes the necessary attachments from point to point. It was evident, quite early in the usage of such sets, that some convenience and other advantages could be gained by incorporating the tubing in a cassette, arranged so as to eliminate the necessity for threading tubing through the operative parts of a machine. There are numerous examples of such disposable cassettes, including a number of tubing cassettes for use with peristaltic pumps. In exchange for the ease of installation, and the reduction in chance of operator error, disadvantages are presented in terms of cost, the need in some instances for complex internal tubing paths that may involve sharp bends, and often the unsuitability of the cassette in the event of any modification, however minor, of the operative parts of the system.

One approach to solving this problem used an irrigation/aspiration system for ophthalmic microsurgery wherein a sterile solution was fed via irrigation tubing through the top of a cassette housing, and around an element providing a backup surface or boss adjacent an opening in the leading edge of the cassette, which was inserted into the machine in edgewise fashion. A clamp was engageable against the tubing and the boss to pinch the tubing and stop the passage of sterile solution out a side edge of the cassette toward a handpiece. Controls available to the surgeon enabled aspiration of non-sterile fluid and tissue from the surgical site back through an aspiration line into a side edge of the cassette and out to an attached waste bag. A length of the tubing close to the front edge of the cassette was exposed adjacent a semicircular surface against which a peristaltic pump could be engaged to withdraw the aspiration fluid. A shunt line from the aspiration line, within the cassette, lead to a vacuum control system opening at the front edge, into which an occluder shaft fit so as to control the differential pressure level available in the aspiration manifold. By operating the controls, the surgeon or technician could open the shunt line to provide instant backflow of irrigation fluid so that cortical material that was caught in the handpiece could be immediately cleaned out.

Another prior approach used an end loaded tubing cassette having a leading edge structure that included operative couplings for a peristaltic pump, an irrigation clamp, a shunt flow clamp, and a vacuum control system opening. The cassette was provided with an intercoupling side opposite the leading edge side, with the internal tubing being principally disposed proximate the intercoupling side, except for a non-sterile aspiration tubing section that passed adjacent the peristaltic pump opening. Sliders engaged in openings in one broad wall of the cassette housing were exposed to the control elements in the machine when the cassette was inserted and spanned the housing interior to the relevant tubing sections. The sliders included operative ends extending toward the intercoupling side, spaced apart from fixed reference surfaces toward which they were directed, with the different irrigation and shunt tubing sections being disposed between them. The sliders formed part of the internal sterile mechanism of the cassette, and in the event of fracturing or penetration of the tubing internally to the cassette, the sterile contents encounter contaminating sources. In addition, the shunt tubing connection intercommunicating with the vent line and the peristaltic pump was not only relatively short but was clamped in this geometry at a point close to the vent line. Only a very short segment of tubing thus was subjected to the drop in pressure and line resonances were not introduced. This arrangement permitted use of the same basic construction to accommodate dimensional and some positional variations in tubing or exterior mechanism. Moreover, the tubing was all held at the intercoupling edge by a stabilizer bar insertable in a section of the cassette housing. The stabilizer bar provided a secure retainer for the tubing ends, to assure against slippage and misplacement. This configuration also minimized the internal length of tubing needed, reducing by a substantial amount the priming volume required. The unit further incorporated means for supporting a replaceable bag into which the aspirated tissue and fluids were passed, and that could be interchanged in the event that capacity was approached during a surgical procedure.

In view of these problems that were not satisfactorily solved by the above described prior approaches, there remains a need for a cassette comprising a cartridge containing an irrigation fluid-flow path and an aspiration fluid-flow path that is adapted to be releasably attached to a surgical instrument wherein when the cartridge is positioned in registry contact with fluid-flow control elements on the instrument, the instrument recognizes the correct positioning of the cartridge and locks the cassette to the instrument.

### SUMMARY

Broadly, the invention is embodied in a system in which reusable or disposable cartridges or an integral cassette may use fluid conduits to carry out aspiration and infusion of fluids. In some embodiments, the system is used during phacoemulsification performed on the natural lens of the eye. The novel cassette receiver assembly of the present invention provides a fluid-connecting fixture for conveniently connecting fluid conduits providing irrigation, aspiration, and pressure sensing in a surgical irrigation-aspiration system that is releasably attachable to a surgical instrument, such as a Phacoemulsification instrument. Further, the system includes one or more sensors, such as switches, for sensing when the reusable or disposable cartridges or integrated cassette are properly positioned on the receiver assembly. Upon sensing proper positioning of the cartridges or cassette, the sensors provide a signal to a control system, typically embodied in a microprocessor based programmable computer that in turn causes the control system to send a signal to one or more locking assemblies. This signal causes the locking assemblies to be activated to firmly, but reversibly, lock the cartridge in place on the receiver assembly. The sensors may include simple pressure switches, or alternatively, may be capable of interacting with designated portions of the cartridge or cassette assembly to sense whether a pre-determined manufacturer produced the cartridge or cassette, and thus determining whether the cartridge or cassette is suitable for use.

Another embodiment comprises a tubing cartridge for engagement with a cartridge-receiving portion of an instrument in which the cartridge-receiving portion of the instrument is a substantially flat surface having a locking mechanism and first and second pinch valves projecting outwardly from the flat surface, the tubing cartridge comprising a housing having a case and a rear cover, the rear cover having first and second valve apertures and a locking aperture within it, and a locking mechanism activator on it, and an irrigation tube having an input end and an output end and a housed portion between them. The housed portion of the irrigation tube is contained within the housing and the input and output ends of the irrigation tube extend from the housing. When the tubing cartridge is placed against the cartridge-receiving portion of the instrument and the locking mechanism activator on the rear cover activates the locking mechanism on the instrument, a first portion of the housed portion of the irrigation tube moves under the first valve aperture and engages the first pinch valve.

Another embodiment provides a pump/sensor cartridge for engagement with a cartridge-receiving portion of an instrument in which the cartridge-receiving portion of the instrument has a substantially flat surface with a locking mechanism and a peristaltic pump head projecting outwardly from the flat surface. The pump/sensor cartridge comprises a housing with a case and a rear cover. The rear cover has a valve aperture and a locking aperture in it, a locking mechanism activator on it. The pump/sensor cartridge further comprising an aspiration tube having an input end and an output end, and a housed portion and a looped portion between the ends in which the housed portion is contained within the housing. The looped portion and the input and output ends of the aspiration tube extend from the housing such that when the pump/sensor cartridge is placed against the cartridge-receiving portion of the instrument and the locking mechanism activator on the rear cover activates the locking mechanism, part of the housed portion of the tube moves under the first valve aperture and engages the first pinch valve, and the looped portion of the tube engages the peristaltic pump head on the instrument.

In still another embodiment, the present invention includes a tubing cartridge having an aspiration tube with an input end, an output end, and a housed length. The housing contains the housed length of the aspiration tubing and the input and the output ends project from the housing.

In yet another embodiment, the present invention includes a tubing cartridge having a reflux tube contained within the housing. One end of the reflux tube is in fluid communication with the irrigation tube, the other end of the reflux tube is in fluid communication with the aspiration tube, and a portion of the reflux tube sits under the second valve aperture and engages the second pinch valve on the instrument.

In a further embodiment, the looped portion of the aspiration tube of the pump/sensor cartridge has a pair of legs connected together by a coupling portion of tubing. In one embodiment, one leg is shorter than the other leg. This provides for improved loading and unloading of the aspiration tube on or off a peristaltic pump. In yet another embodiment, the looped portion is substantially U shaped, wherein one of the legs of the U is shorter than the other leg. In still another embodiment, the legs of the looped portion are substantially equal in length.

Other features and advantages of the present invention will become more apparent from the following detailed description, taken in conjunction with the accompanying drawings, that illustrate, by way of example, the principles of the invention.

### BRIEF DESCRIPTION OF THE DRAWINGS

FIG. 1 is a block diagram of an embodiment of a surgical apparatus in which the apparatus includes fluid-flow control means operable for controlling the flow of an irrigation fluid to a handpiece and the flow of aspirant away from the handpiece.

FIG. 2 is a schematic diagram illustrating the flow of fluid to and from an eye in a phacoemulsifier apparatus.

FIG. 3 is a perspective view of a reusable tubing cartridge attachable to a surgical instrument for directing the flow of irrigation fluid to a surgical handpiece.

FIG. 4 is an exploded view of the reusable tubing cartridge of FIG. 3.

FIG. 5 is a perspective view of a disposable tubing cartridge attachable to a surgical instrument for directing the flow of irrigation fluid to a surgical handpiece.

FIG. 6 is an exploded view of the disposable tubing cartridge of FIG. 5.

FIG. 7 is a perspective view of a pump/sensor cartridge attachable to a surgical instrument for directing the flow of aspiration fluid to or from a surgical handpiece.

FIG. 8 is an exploded view-of the pump/sensor cartridge of FIG. 7.

FIG. 9 is a perspective view of a pump/sensor cartridge attachable to a surgical instrument for directing the flow of irrigation and aspiration fluid to and from a surgical handpiece.

FIG. 10 is an exploded view of the pump/sensor cartridge of FIG. 9.

FIG. 11 is a plan view of a cartridge-receiving portion of an instrument adapted to receive the tubing cartridge and pump/sensor cartridge of one embodiment of the present invention.

FIG. 12 is a plan view of a cartridge-receiving portion of FIG. 11 with the tubing and pump/sensor cartridges mounted in place and locked on to the cartridge-receiving portion.

FIG. 13 is a cross-sectional side view of the assembly depicted in FIG. 12 showing integral tubing and pump/sensor cassette mounted to the cartridge-receiving portion.

FIG. 14 is a plan view of a printed circuit board disposed in the case of pump/sensor cartridge and having a pressure sensor that monitors the pressure in the lumen of the aspiration tubing.

### DETAILED DESCRIPTION

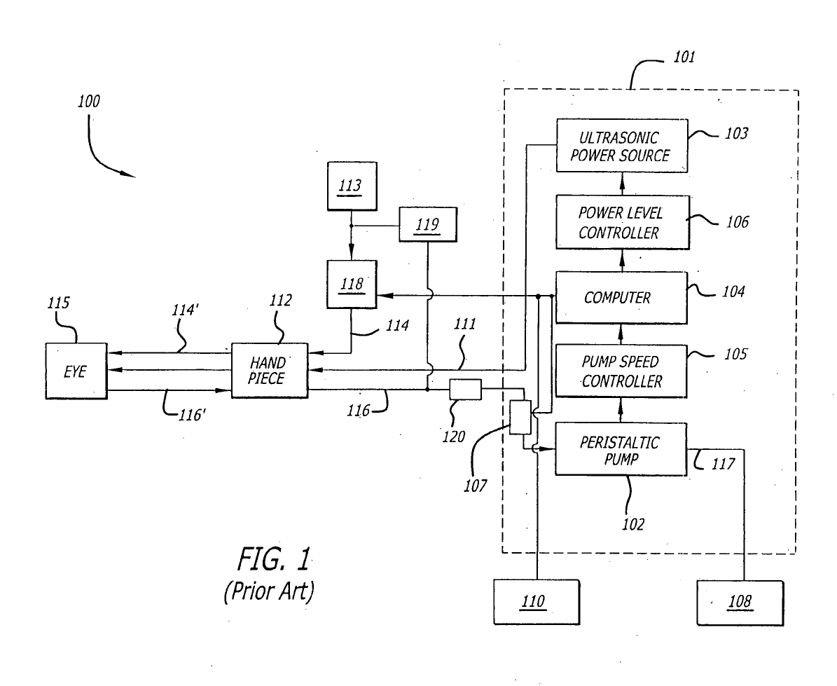

Turning first to FIG. 1, a medical apparatus 100 is illustrated in block diagrammatic form. Examples of suitable medical apparatus include for example phacoemulsification apparatus. But other apparatuses that need similar fluid handling characteristics are within the scope of this disclosure. The apparatus 100 includes a control unit 101 (FIG. 1), a variable speed peristaltic pump 102 that provides a vacuum source, a source of pulsed ultrasonic power 103, and a microprocessor-based programmable computer 104 that provides control outputs to pump-speed controller 105 and ultrasonic power level controller 106. The components of the apparatus 100 need not be integral to the apparatus. For instance, the variable speed peristaltic pump 102 could be a separate unit connected to the apparatus 100 with control or power lines. A suitable memory or other means for storing information, such as random access memory, read only memory, or magnetic or tape data storage devices, may be operably connected to the programmable computer. The phacoemulsification apparatus 100 may also include input means such as a keyboard, card reader, push buttons, touch screen, or other user interface for inputting data and treatment and operational parameters to the apparatus to program or control the operation of the phacoemulsification apparatus 100. The apparatus 100 may also include a communication device, such as a data port, which may be a COM port, a universal serial bus, or an IEEE 1394 port, or other device for establishing communication with a network or external computer or storage device.

A vacuum sensor 107 provides a signal to computer 104 establishing the vacuum level on the input side of a peristaltic pump 102. Reversing the peristaltic pump 102 may provide suitable venting. A footswitch pedal 110 is provided to enable a physician to control irrigation fluid, aspiration rate, and ultrasonic power level as will be described.

In one embodiment, the instrument control unit 101 supplies ultrasonic power on line 111 to a phacoemulsification handpiece 112. An irrigation fluid source 113 is fluidly coupled to handpiece 112 through line 114 and from the handpiece 112 to the eye 115 through irrigation line 114′. The flow rate of the irrigating fluid contained in fluid source 113 is driven by gravity and is usually adjusted by raising and lowering the fluid source 113 with respect to the handpiece 112. The irrigation fluid and ultrasonic power are applied by handpiece 112 to a patient's eye 115. Aspiration of irrigating fluid and fragmented lens tissue from the eye 115 is achieved by means of a peristaltic pump 102 housed within the control unit 101 through aspiration lines 116 and 116′. A programmable computer 104 responds to actual vacuum levels in the input line 116 by measuring the signal from vacuum sensor 107 and then controlling the flow rate of the peristaltic pump 102.

If the handpiece aspiration line 116′ becomes occluded, vacuum sensor 107 will show an increased vacuum level. The computer 104 preferably includes operator-settable limits for aspiration rate, vacuum level, and the level of ultrasonic power delivered to the handpiece 112 and hence the eye 115. When occlusion of the handpiece aspiration line 116′ causes the vacuum level sensed by vacuum sensor 107 to reach a predetermined level, the programmable computer 104 instructs pump-speed controller 105 to change the speed of the peristaltic pump 102 that, in turn, changes the aspiration rate.

In the event that aspirated material occludes the aspiration line, the speed of the peristaltic pump 102 can either be increased or decreased (or the level of ultrasonic power varied) to restore flow though the aspiration line. When the occluding material is broken up or otherwise removed, the vacuum sensor 107 shows a drop in vacuum level, causing the programmable computer 104 to change the speed of peristaltic pump 102 to an unoccluded operating speed. The programmable computer 104, via the irrigation fluid source 113, may further provide a reflux fluid supply to the aspiration line 116′ by switching the flow of irrigation fluid to the aspiration line 116 by means of fluid-flow controlling valves 118, 119, and 120. The reverse flow (reflux) of irrigation fluid through the aspiration line 116 and 116′ and the aspiration channel within the handpiece 112 may be used to remove such an obstruction.

Current practice favors systems that separate fluids employed in the performance of a surgical procedure from the instrument to avoid contamination and facilitate using the instrument in multiple procedures (involving different patients) without substantial delay between them. The present invention provides a fluid-flow control system comprising a cartridge that contains an irrigation fluid-flow path and an aspiration fluid-flow path. The cartridge is adapted to be releasably attached to a surgical instrument 101 such that when the cartridge is positioned in registry and contact with fluid-flow control elements on the instrument, the instrument recognizes the correct positioning of the cartridge and locks the cassette to the instrument.

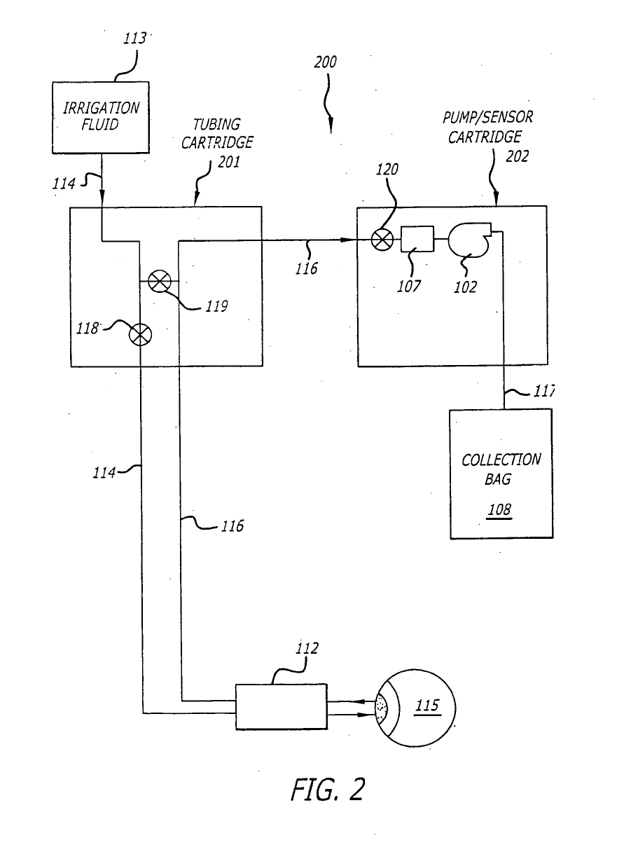

With reference to FIG. 2, an irrigation/aspiration flow system and flow control elements are shown at 200 in schematic view. The pump 102 and the fluid-flow control valves 118-120, disposed to seal or open the fluid-flow lines 114 and 116, are part of the instrument 101 to which the irrigation/aspiration flow system 200 attaches. The portion of the fluid-flow system comprising the present invention includes a tubing cartridge 201 and a pump/sensor cartridge 202, both of the cartridges 201 and 202 being adapted to releasably and lockingly engage the instrument 101 (FIG. 1) such that when the cartridges 201 and 202 lockingly engage the instrument 101, fluid control valves 118-120 control the flow of fluid through the portion of the fluid lines 114 and 116 housed within the cartridges.

Irrigation fluid flows from a reservoir 113 through irrigation tubing 114 through valve 118, which is used to control the fluid-flow rate, through the handpiece 112, and into the eye 115. Aspiration pump 102 engages aspiration fluid tubing 116, as will be explained in more detail below, to create a vacuum in aspiration line 116. This vacuum aspirates fluid and debris, which is produced during the emulsification of the natural lens of the eye, from eye 115 through handpiece 112. Valve 120 is used to set vacuum parameters during initial system priming and testing. Sensor 107 monitors the vacuum in the aspiration fluid line 116, and, if the vacuum rises beyond a predetermined limit, indicating that a blockage of the handpiece 112 has occurred, signals the programmable computer 104. The computer 104 in turn may signal valve 119 to open and valve 120 to close. This allows irrigation fluid to flow directly into aspiration fluid line 116. This influx relieves the vacuum in line 116 and assists to clear the line blockage by reversing fluid flow through the handpiece 112.

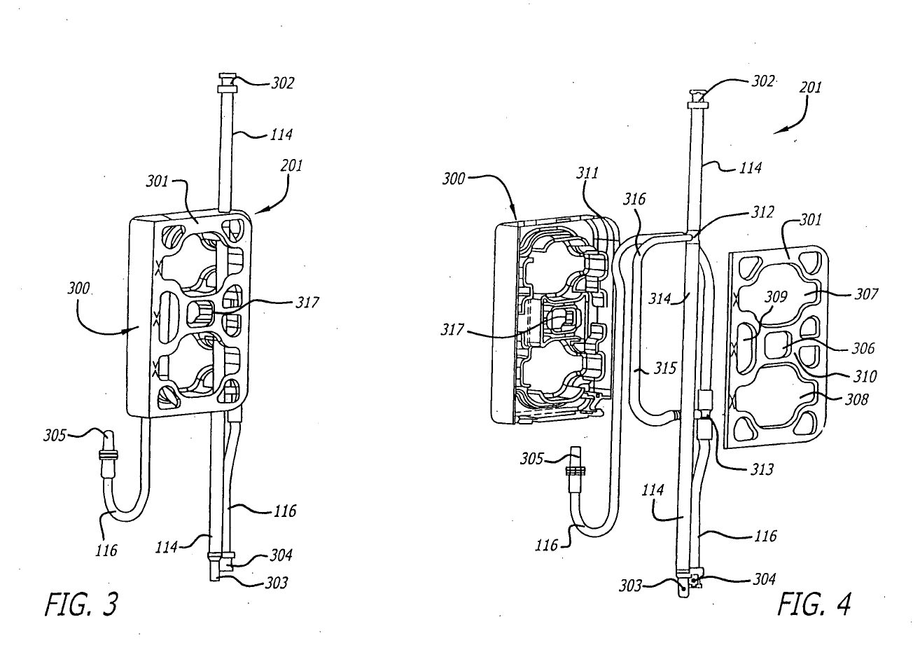

The tubing cartridge 201 and the pump/sensor cartridge 202 may be fabricated either as separate cartridges or combined within a single cassette adapted for attachment to an instrument. FIG. 3 is a perspective view of a reusable tubing cartridge 201 adapted to be attached to a cartridge-receiving portion of a surgical instrument such as shown at 101 in FIG. 1. The tubing cartridge 201 directs the flow of irrigation fluid to a surgical handpiece wherein certain fluid-flow characteristics can be controlled by the instrument to which the tubing cartridge 201 is attached. The tubing cartridge 201 includes an irrigation fluid tubing 114 defining an irrigation fluid-flow path and an aspiration fluid tubing 116 defining an aspiration fluid-flow path contained within the tubing cartridge 201. The irrigation fluid tubing 116 has a quick-release Luer-type fitting 302 on the intake end and a similar quick-release fitting 303 on the output end. The aspiration tubing 116 also has a Luer-type fitting 304 on the intake end and a similar quick-release fitting 305 on the output end.

FIG. 4 is an exploded view of the reusable tubing cartridge 201 of FIG. 3 showing the routing of the respective irrigation and aspiration tubing through the case 300 of the cartridge 201. The irrigation and aspiration tubing, 114 and 116 respectively, are routed through and housed within a plastic case 300 having a rear cover 301. The rear cover 301 includes four strategically placed cutouts 306-309. When the cutouts 306-308 are properly aligned with matingly engaging valve controls (not shown) projecting from a cartridge-receiving portion of the instrument, the lock actuator portion 310 of cover 301 provides means for activating a sensor switch on the instrument 101. The sensor switch may take the form of a simple switch, or it may take the form of a more complex sensor. For example, the sensor may be a detector that interacts with the lock actuator portion 310 in such a way that the sensor is capable of detecting a unique identifier mounted on the lock actuator portion 310 that identifies the origin, manufacturer, and/or type of the cartridge or cassette. Such an identifier, for example, may include a bar code mounted on the lock actuator portion 310, sending a signal to the programmable computer 104. In another embodiment of the invention, the identifier may include a Radio Frequency Identification (RFID) tag mounted anywhere on or otherwise associated with the cartridge or cassette. In another embodiment of the invention, the identifier may include a resistor mounted on the lock actuator portion 310. In yet another embodiment of the invention, the identifier can be stored in a Read Only Memory (ROM) or other memory device mounted to the lock actuator portion 310. The signal is then analyzed by the programmable computer 104 to determine whether the cartridge or cassette that has been mounted on the receiver apparatus is appropriate for use (e.g., compatible) with the apparatus based on the value (bar code, RFID, resistance, etc.) of the identifier. In the event that the cartridge or cassette is not appropriate, the programmable computer 104 may send a signal to the locking mechanism, described in more detail below, preventing the locking mechanism from engaging the cassette or cartridge. The computer 104 may also provide a visual or auditory signal to the operator that the cartridge or cassette is not appropriate for use with the apparatus, prompting the operator to replace the cartridge or cassette. It will be appreciated by one of ordinary skill in the art that the unique identifier can be located in different locations of the cartridge or cassette.

In an embodiment in which the tubing cartridge 201, the pump/sensor cartridge 202, or a cassette combining the cartridges 201 and 202 are not reusable, the identifier can vary to indicate whether a cartridge is new and appropriate for use or used and inappropriate for use. In one embodiment, the sensor or other device can change a first setting indicating appropriateness for use to a second setting indicating inappropriateness for use. Thereafter, the computer 104 will prevent the cartridge from being reused when the identifier is set to the second setting. Accordingly, the variable identifier can prevent use of a used cartridge or cassette that might be contaminated from a previous use or is otherwise inappropriate for use.

In one embodiment, the unique identifier may include a variable resistor, such as a rheostat, with at least two settings. In another embodiment of the invention, the identifier may be included in a memory device such as flash memory and can be changed by the sensor or other device after using the cartridge.

The valve controls that project into the cartridge through the cutouts 307 and 308 are operable for pinching either the irrigation tubing or the aspiration tubing or both, the choice of activated valves being controlled by a user-generated command to the instrument to which the cartridge 201 is attached. Such user-generated commands typically originate in the foot control 110 that is actuated by a user of the apparatus 100. Actuation of the foot control 110 sends signals to the programmable computer 104 that in turn provides appropriate control signals to the necessary valves to carry out the operator's commands.

The case 300 has molded-in tubing guides that serve to route and support the tubing within the cartridge. A reflux tube 316 is connected to a side arm of a first tee connector 312 disposed in the irrigation tubing 114 and a side arm on a second tee connector 313 disposed in the aspiration tubing 116. The reflux tubing 316 provides fluid connection between the irrigation tube 114 and the aspiration tube 116. When the cartridge 201 is correctly positioned against the cartridge-receiving portion of the instrument 101, pinch valves 118 and 119 on the instrument 101 (FIG. 1) engage the irrigation tubing 114 and the reflux tubing 316 at pinch points 314 and 315 respectively. In some embodiments the pinch valves are operable in the plane parallel to the instrument housing, e.g. the pinching motion is substantially parallel to the instrument housing. The cartridge includes a locking flange 317 disposed on the case behind and adjacent to cutout 306 in the cover 301. A lock activator portion 310 of the cover 301 presses against and activates a locking means in the instrument causing a locking mechanism within the instrument to lockingly engage the locking flange 317, signaling the instrument 101 that the tubing cartridge is registerably attached and activating the pinch valves 118 and 119. Under normal operating conditions, pinch valve 118 is open, permitting irrigation fluid to flow through the irrigation tubing and pinch valve 119 is closed, preventing irrigation fluid from entering the aspiration tubing.

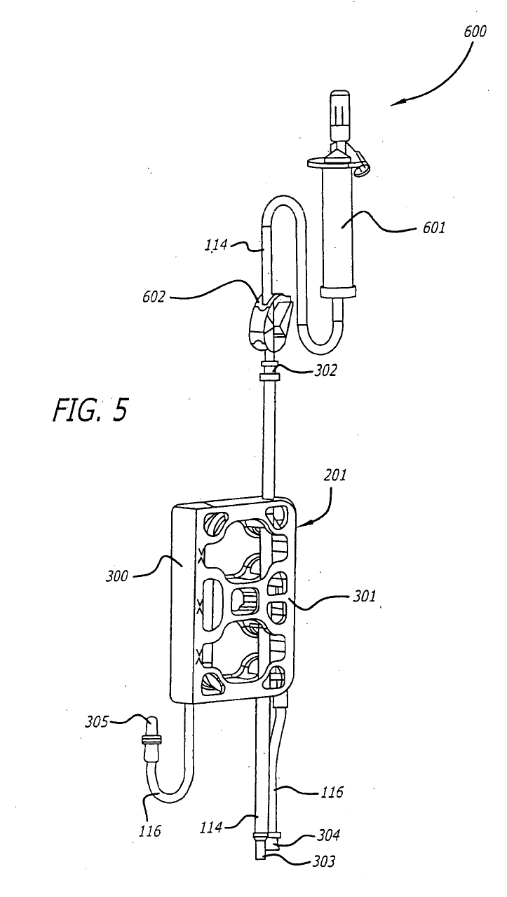

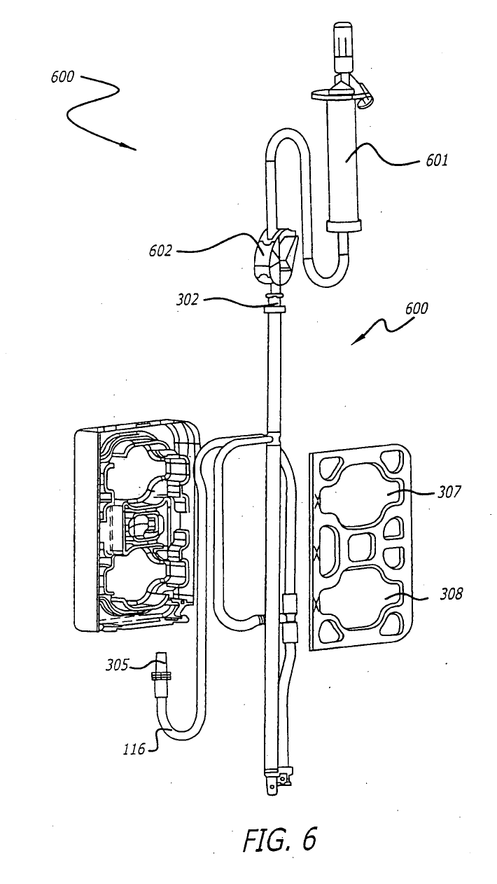

A disposable tubing cartridge 600 is shown in rear perspective view in FIG. 5 and exploded rear perspective view in FIG. 6. The disposable tubing cartridge 600 is identical to the reusable tubing cartridge 201 but further includes a drip assembly 601 and fluid drip control means 602 disposed next to the input end 302 of the irrigation tubing 114. The case 300 and rear cover 301 may be molded using a rigid or semi-rigid plastic. The plastic should be rigid enough to maintain the tubing in position as the cassette or cartridge is mounted onto the instrument. The tubing used for the irrigation and aspiration fluid flow is preferably made from an extensible elastomer such as a silicone elastomer, although another tubing material having suitable characteristics that allows the tubing to function as part of the aspiration pump may be used.

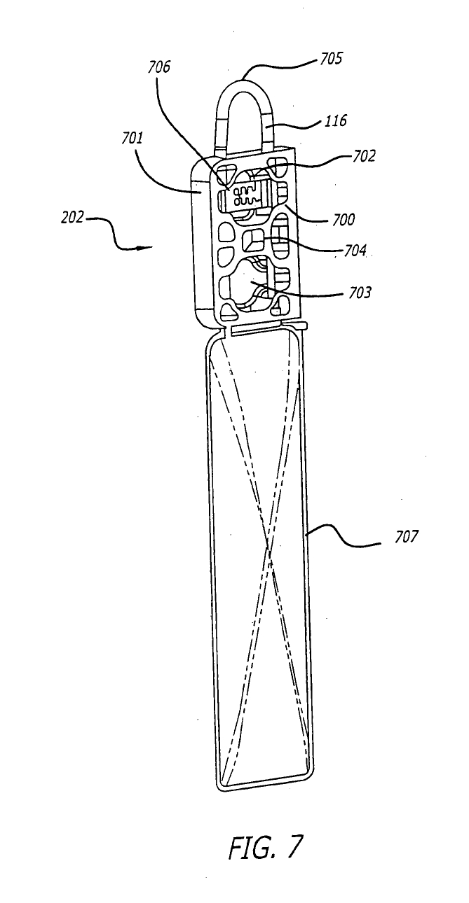

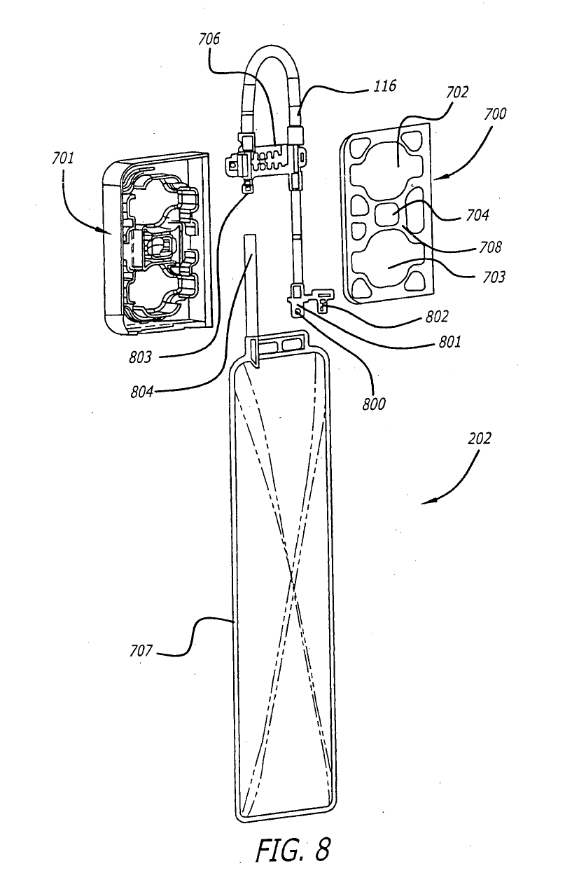

The tubing cartridge 600 is intended for use with a pump/sensor cartridge that is also adapted to be attached to a cartridge-receiving portion of the instrument 101 in a manner similar to the tubing cartridge 201 discussed above. The output end of the aspiration tube 116 projecting from the tubing cartridge 201 (FIGS. 4 and 5), on both the reusable and disposable tubing cartridges, includes a Luer-type fitting 305. The Luer-type fitting 305 provides means for facile connection of the aspiration tubing 116 to a pump/sensor cartridge 202, shown in greater detail in FIGS. 7 and 8. As with the tubing cartridge 201, the pump/sensor cartridge 202 includes aspiration tubing 116 routed through, and supported by, a plastic housing. The pump/sensor cartridge housing comprises a rear cover 700 having cutouts 702-704 attached to a case 701. The aspiration tube 116 has an input end 800 with a Luer-type fitting 801 that matingly engages the Luer-type fitting 305 on the output end of the tubing cartridge aspiration tube. A plug 802 provides sealing engagement with the Luer-type fitting 801 when the fitting 801 is not engaging Luer-type fitting 305.

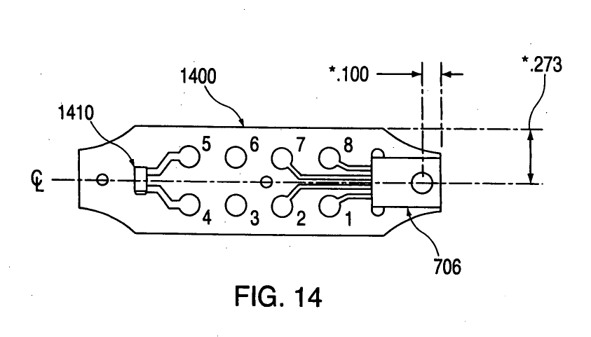

A loop portion 705 of the aspiration tubing 116 projects above the pump/sensor cartridge housing. The loop portion 705 is dimensioned to circumferentially engage a peristaltic pump head projecting from the cartridge-receiving portion of the instrument. A pressure sensor 706 disposed in the case 701 monitors the pressure in the lumen of the aspiration tubing 116 upstream of the loop portion 705. A printed circuit board on which the pressure sensor 706 is disposed will be discussed in further detail below in conjunction with FIG. 14. The downstream end 803 of the aspiration tubing 116 releasably engages collection tubing 804, which connects to an aspirate collection bag 707.

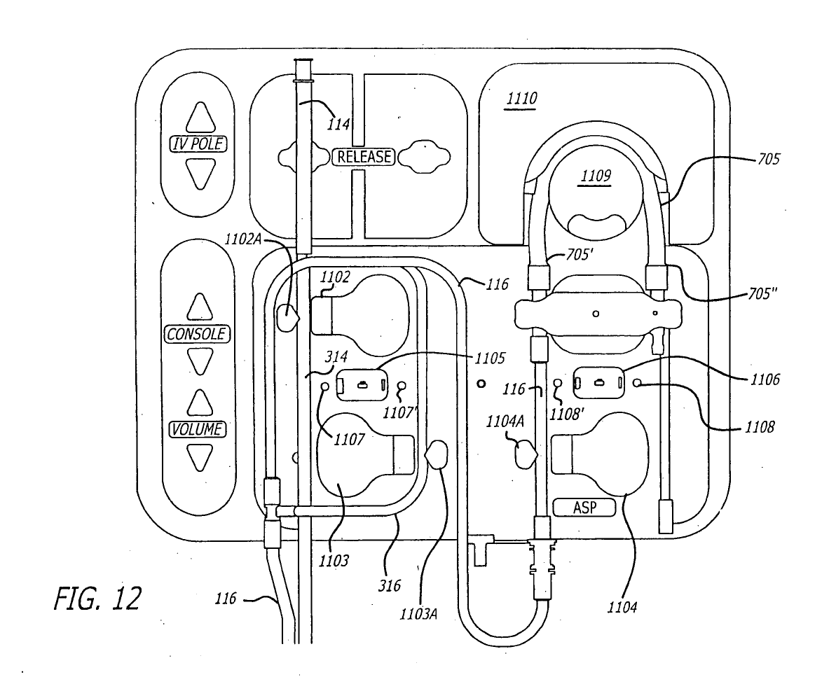

While the loop portion 705 of the aspiration tubing 116 is shown in FIGS. 7-10 and 12 as being generally U-shaped, loop portion 705 may also be formed such that one leg of the U is shorter than the other leg. In one embodiment of the present invention, leg 705′ is shorter than leg 705″ (FIG. 12). This arrangement provides an offset in length of the two legs of loop portion 705 that facilitates engagement and disengagement of the loop portion 705 with the peristaltic pump. In some embodiments, such an arrangement provides for quieter operation of the pump/tubing combination, less stretching of the tubing during loading and unloading, and better stability of the tubing during operation of the pump.

A further advantage of some embodiments of the present invention is that the unique arrangement of tubing and latching mechanisms act together to provide a spring force that assists in ejecting the cassette and/or cartridge from the receiver assembly. When the cassette or cartridge is pushed down onto the receiver assembly, and the lock activator switches engage, causing the cartridge or cassette to lock onto the receiver, the tubing arrangement and the valves cause portions of the tubing to be offset such that the offset portions act as springs and provide a force that tends to oppose the force of the latching mechanism. When the solenoids are energized, opening the latching tongues to release the cartridge or cassette from the receiver, this spring force tends to force the cartridge or cassette away from the receiver, facilitating removal of the cassette or cartridge. Another advantage of this arrangement is that by biasing the cassette or cartridge away from the receiver, the force provided by the tubing also pushes the cassette or cartridge off the activator switch or switches, helping to ensure that inadvertent handling of the cassette or cartridge does not result in re-engagement of the cassette or cartridge on the receiver through an inappropriate activation of one or more of the activator switches.

When the pump/sensor cartridge is properly mounted on the receiver assembly, valve controls protrude through cutouts 702-703. The valve controls are operable to pinch the aspiration fluid tubing 116 to control fluid flow. Pump/sensor cartridge 202 may also include a lock actuator portion 708 located on rear cover 700 designed to interact with a sensor (not shown) disposed on the receiver assembly. The design and operation of the lock actuator portion 708 and sensor are similar to the lock actuator portion 310 and the sensor described above in reference to the tubing cartridge 201.

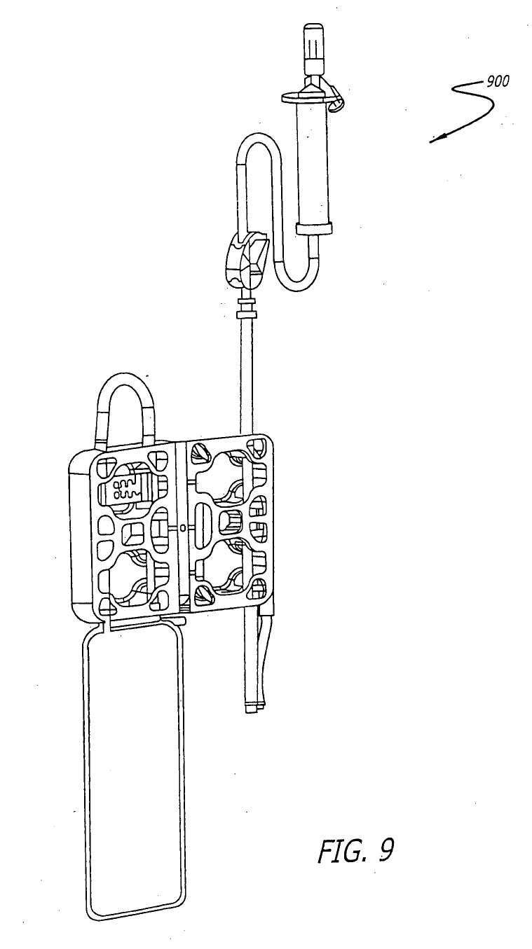

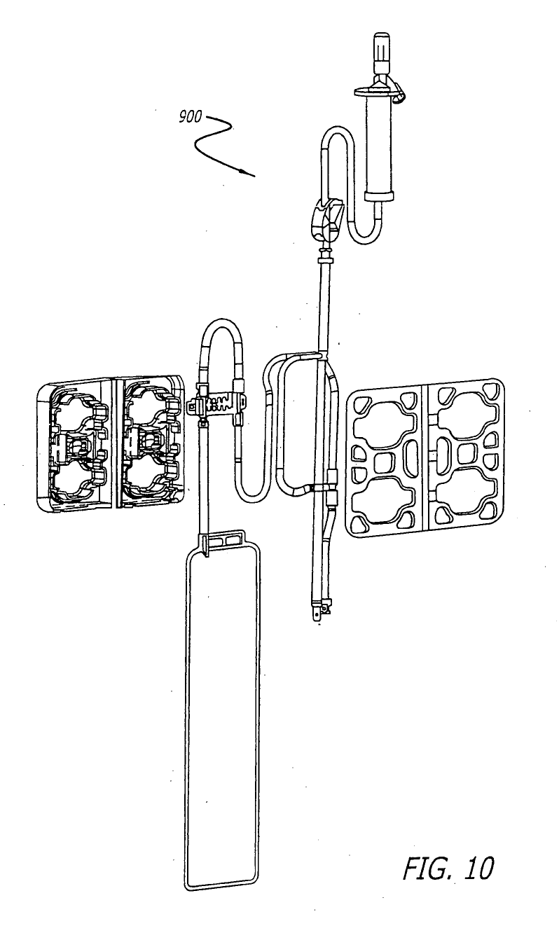

The pressure sensor 706 is preferably a pressure sensitive transducer that produces an electrical signal that is proportional to the pressure of aspiration fluid in the aspiration tube upstream of the peristaltic pump head. Such pressure sensitive transducers are commercially available, for example, Motorola Part No. MPX 2301 DT1. In some embodiments the case and rear cover of both the tubing cartridge and the pump/sensor cartridge are identical to reduce the production cost of the cartridges. In addition, the tubing cartridge 201 and pump/sensor cartridge 202 can be integrated and manufactured as a single cassette, indicated at numeral 900 in FIGS. 9 and 10.

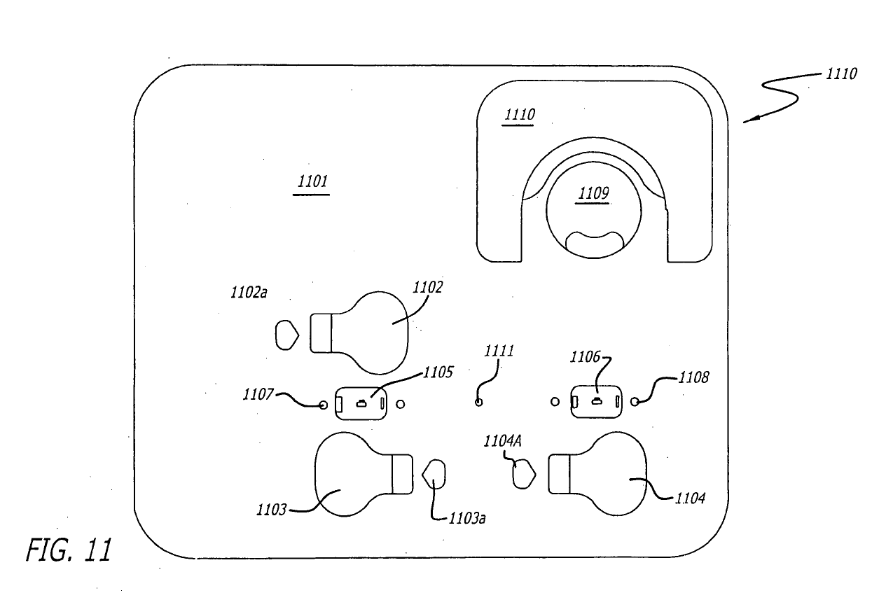

The cartridge-receiving portion of the instrument indicated at 1100 in FIG. 11 comprises a flat surface 1101 having a plurality of pinch valves, locking mechanisms, lock activators, and a peristaltic pump head projecting outwardly from the surface. A first pinch valve 1102 includes an anvil portion 1102a separated by the diameter of the irrigation tubing in the tubing cartridge. A second pinch valve 1103 and valve anvil 1103a pinch a portion of the reflux tube in the tubing cartridge passing between them. A third pinch valve 1104 and pinch valve anvil 1104a control the flow of fluid through the aspiration tube housed within the pump/sensor cartridge. In some embodiments these pinch valves are operable in a plane substantially parallel to the cartridge-receiving portion of the instrument, in a plane substantially parallel to the rear cover of the cassette of the cartridge, or both. Depressing the lock mechanism activator buttons 1107 and 1108 activates locking mechanisms 1105 and 1106 to loc the tubing cartridge and the pump/sensor cartridge respectively to the instrument. This arrangement allows use of either the disposable or reusable tubing and pump/sensor cartridges shown in FIGS. 3-8 or the integral cassette combining tubing and pump/sensor functions as shown in FIGS. 9 and 10. The fluid-flow path, defined by the irrigation tube, aspiration tube, and reflux tube, in combination with the pinch valves is illustrated in FIG. 12.

Depressing the lock mechanism activator button 1111 can also activate locking mechanisms 1105 and 1106. While the lock mechanism activator buttons 1107 and 1108 may be located as indicated by 1107 and 1108, alternative embodiments locate the lock mechanism activator buttons as indicated by 1107′ and 1108′.

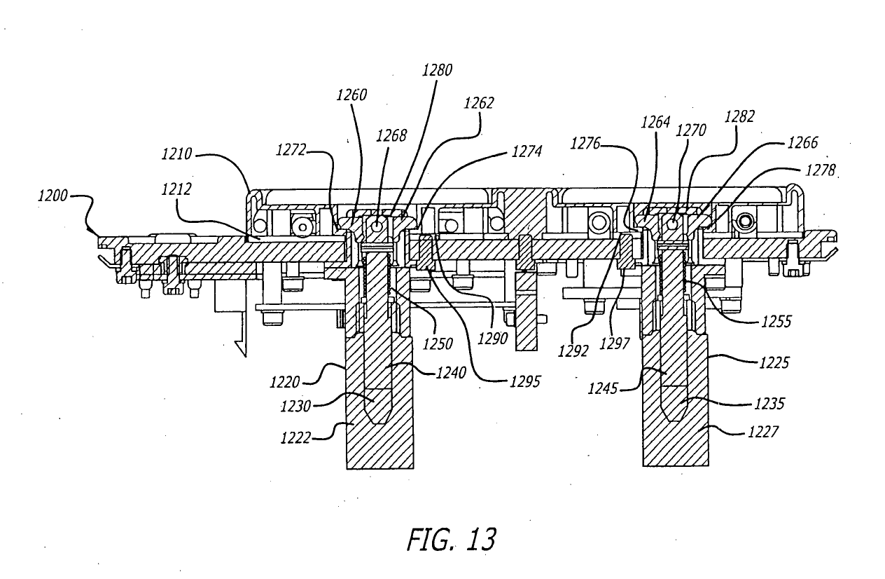

Referring now to FIG. 13, a more detailed description of the locking mechanisms for holding the disposable or reusable cartridges or integral cassette in place on the cartridge-receiving portion will be described. As shown in cross-section, the cartridge-receiving portion 1200 includes locking mechanisms 1220 and 1225. These receive and lock the cartridges or cassette in place on the cartridge-receiving portion 1200. These mechanisms may operate either individually to lock an individual pump/sensor cartridge or tubing cartridge in place, or they may operate together to hold an integral tubing/pump/sensor cassette in place. For convenience, the integral tubing/pump/sensor cartridge 1210 is shown mounted in place on cartridge-receiving portion 1200, but those skilled in the art will understand that the description of the features of FIG. 13 applies equally to embodiments employing individual tubing and pump/sensor cartridges.

The locking mechanisms 1220 and 1225 each have a locking portion that extends above the top surface of cartridge-receiving portion 1200. When the cartridges or cassette are properly aligned on the cartridge-receiving portion 1200 and when the locking mechanism is activated, a portion of the locking mechanisms 1220 and 1225 protrude through cut-outs in the cartridges or cassette to engage the back cover of the cartridges or cassettes to lock the cartridges or cassette in place. Alternatively, locking mechanisms 1220 and 1225 may comprise a system of cams that lock against a wall defined by the cutout in the cartridge or cassette.

Each of locking mechanisms 1220 and 1225 include a bore 1230 and 1235 that extends longitudinally through the locking mechanism body 1220 and 1225. Locking mechanism bodies 1220 and 1225 include electromagnetic or pneumatic solenoids 1222 and 1227 that are configured to act upon pistons 1250 and 1255 respectively. Pistons 1240 and 1245 are biased in upward position by springs 1250 and 1255. Locking mechanism 1220 includes locking tongues 1260 and 1262, and locking mechanism 1225 include locking tongues 1264 and 1266.

The operation of locking mechanisms 1220 and 1225 will now be described. For brevity, only the operation and structure of locking mechanism 1220 will be described, it will be apparent that the structure and operation of locking mechanism 1225 is substantially similar to that of locking mechanism 1220. But one of ordinary skill in the art will understand that while the operation of the locking mechanisms 1220 and 1225 are similar, some modifications may be necessary to the structure of one or another of locking mechanisms 1220 and 1225 to accommodate particular design requirements of the cartridges or cassette without departing from the scope of the intended invention.

Latching tongues 1260 and 1262 are designed to engage locking surfaces 1272 and 1274. These surfaces are formed into the tubing portion of integral tubing/pump/sensor cassette 1210. Similarly, latching tongues 1264 and 1266 are designed to engage locking surfaces 1276 and 1278 and formed into the pump/sensor portion of integral tubing/pump/sensor cassette 1210. Latching tongues 1260 and 1262 are pivotally mounted on axle 1268. Axle 1268 extends through a bore 1280 and 1282 formed by substantially U-shaped channel or socket disposed at the top of piston 1250. Locking mechanism 1220 includes a housing having an upper end 1280 that extends above the surface of the cartridge-receiving portion 1200. The housing includes a pair of diametrically opposed openings located in the upper end of the housing 1280, as well as an inner surface disposed at the top of the bore 1230.

Tongues 1260 and 1262 extend through the openings in the upper end 1280 of the housing to engage latching surfaces 1272 and 1274. The tongues, axle, and internal surface of the upper end 1280 of the housing cooperate so that when piston 1240 is biased in its upward direction, the upper surfaces of tongues 1272 and 1274 press upon the internal surface of the upper end 1280 of the housing and thus are maintained in a locked position to engage latch surfaces 1272 and 1274 to hold the cartridge or cassette in place on cartridge-receiving portion 1200.

When a cartridge or cassette is to be mounted on cartridge-receiving portion 1200, an operator will typically initiate the mounting sequence by actuating a push button, touch screen control, or other input means that sends a signal to the programmable computer 104 indicating that a cartridge or cassette is ready to be mounted on cartridge-receiving portion 1200. The programmable computer 104 then provides a signal to the locking mechanism 1220 that energizes the solenoid 1222. When solenoid 1222 is energized, the solenoid generates an electromagnetic field that acts upon piston 1240 moving it downward in bore 1230. The downward motion of piston 1240 pulls the end of latching tongues 1260 and 1262 mounted on axle 1268 downwards. The surfaces of tongues 1260 and 1262 contact the upper surface of cartridge-receiving portion 1200 and contact the latching surfaces 1272 and 1274 of the cartridge or cassette. These tongues are rounded; the downward motion of piston 1240 causes the tongues 1260 and 1262 to rotate about axle 1268 into an upwards, unlocked position allowing the upper end of the piston 1240, including the tongues 1260 and 1262, to be drawn into bore 1230. As the tongues 1260 and 1262 rotate upwards and are drawn into bore 1230, the tongues 1260 and 1262 are drawn back through the openings in the upper end 1280 of the housing. In this configuration, the diameter of the housing extending upwards from the surface of the cartridge-receiving portion 1200 is slightly less than a corresponding opening in the rear cover of the cartridge or cassette, allowing the housing to extend through the opening when the cartridge or cassette is properly aligned on the surface of the cartridge-receiving portion 1200.

When integral tubing/pump/sensor cassette 1210 is placed upon cassette-receiver portion 1200, the cassette presses down on at least one switch 1295. As shown, cassette-receiving portion may also include a second switch 1297. These switches are located such that they activate when individual tubing or pump sensor cartridges are mounted, or when an integral cassette is mounted. Alternatively, the switch may comprise a sensor for an identifier on the cartridge or cassette. For example, the sensor may be a light sensing element that detects a bar code or other identifier formed on the rear cover of the cartridge or cassette, or the sensor may be an electromagnetic (EM) sensor capable of sensing an embedded chip or transducer, such as are used in so-called “smart cards” or the cartridge or cassette may even include an embedded processor that may be queried by the sensor/transducer to determine the identity of the cartridge or cassette. In another embodiment, the EM sensor senses a resistor mounted on or in the cartridge or cassette. In another embodiment of the invention, a RF sensor senses an RFID tag attached to the cartridge or cassette. Instruments incorporating this feature will be capable of determining whether a cartridge or cassette is appropriate for use with the instrument. For example, the sensor may be configured to detect whether the cartridge or cassette, if reusable, has been sterilized or otherwise inspected and approved for reuse, or the sensor may be configured to detect the origin or manufacturer of the cartridge or cassette. It will be obvious to those skilled in the art that the described switch or sensor will provide signals to the programmable computer that will, in accordance with an appropriate program, carry out the desired determination of appropriateness of the cartridge or cassette.

In another embodiment in which the cartridge or cassette is disposable (not reusable), the identifier can be varied to indicate whether the cartridge or cassette is new or has been used. For example, the identifier can include a variable resistor (e.g., rheostat) or the identifier can be stored in a memory device, such as a flash memory. The sensor or other instrument can then be configured to modify the identifier after use of the cassette or cartridge. In addition, the sensor can be configured to determine if the identifier has been set to indicate that the cassette or cartridge has been used. The programmable computer 104 can then prevent the use of the cassette or cartridge, thereby preventing contamination by using a used cassette or cartridge. Alternatively, the sensor can be configured to include a fusible linkage that the programmable computer 104 may supply with a voltage sufficient to open the linkage after the cassette or cartridge has been installed so that the cartridge or cassette cannot be reused.

When the switch 1295 is actuated, it provides a signal to the programmable computer 104 of the instrument 101. The programmable computer 104 may then send a signal to the locking mechanism 1220, de-energizing the solenoid 1222. When solenoid 1222 is de-energized, piston 1240 is biased upwards by spring 1250. This upwards bias causes piston 1240 to move upwardly in bore 1230, forcing tongues 1260 and 1262 against the inner surface of the upper end 1280 of the housing, causing tongues 1260 and 1262 to pivot about axle 1268 and extend through the openings in the sides of the housing to engage latching surfaces 1272 and 1274, locking the cartridge or cassette in place on cartridge-receiving portion 1200.

When the cartridge or cassette is mounted in the cartridge or cassette receivers, the tubing in the cartridge or cassette may be slightly compressed due to being slightly bent by the interaction of the various parts of the cartridge and cassette and the locking mechanisms. This compression results in the tubing acting as a spring that provides a force opposing the latching tongue clamping force. When the solenoids or pneumatic actuators are energized releasing the locking mechanisms, the spring force provided by this compression acts to push the cartridge or cassette off the activator switches, and aids in removing the cartridge and/or cassette from the receivers.

It will be understood by those skilled in the art that locking mechanisms 1220 and 1225 may be energized simultaneously when an integral tubing/pump/sensor cassette is used, or they may be energized and locked individually when separate tubing cartridges and pump/sensor cartridges are used. Thus, it may be desirable to include more than one cassette lock mechanism switch (not shown) on cassette-receiver portion 1200. For example, two switches may be used and mounted cassette-receiver portion 1200 such that they would, be energized separately in the case where separate tubing cartridges and pump/sensor cartridges are used or simultaneously where an integral tubing/pump/sensor cartridge is used. Additionally, appropriate sensors may be mounted on cassette-receiver portion 1200 in cooperation with locking mechanisms 1220 and 1225 to sense the position of latching tongues 1260, 1262, 1264 and 1266 to ensure that when an integral tubing/pump/sensor cartridge is used, the integral cartridge sits substantially flat against cassette-receiving portions 1200 so that all fluid lines and locking mechanisms are correctly engaged. In the event that incorrect mounting is sensed, a warning could be communicated to the instrument operator indicating that the cassette needed to be remounted before use. Alternatively, the controller of the device could be locked out to prevent operation of the machine until the signals from the sensors indicated that the cassette or cassettes had been properly mounted.

FIG. 14 is a plan view of a printed circuit board (PCB) 1400 disposed in the case of the pump/sensor cartridge and having a pressure sensor 706 that monitors the pressure in the lumen of the aspiration tubing. The pressure sensor 706 is preferably a pressure sensitive transducer that produces an electrical signal that is proportional to the pressure of aspiration fluid in the aspiration tube upstream of the peristaltic pump head. The electrical signal can be measured at points 1, 2, 7, and/or 8 on the PCB 1400. The PCB 1400 also includes a resistor 1410 having a fixed resistance that acts as an identifier of the origin, manufacturer and/or type of cartridge. A sensor on the control unit 101 measures the resistance of the resistor 1410 between points 4 and 5 of the PCB 1400. The resistance is then analyzed by the programmable computer 104 to determine whether the cartridge or cassette that has been mounted on the receiver apparatus is appropriate for use (e.g., compatible) with the apparatus based on the value (resistance) of the resistor 1410 as sensed by the sensor. In the event that the cartridge or cassette is not appropriate, the programmable computer 104 may send a signal to the locking mechanism, as described above, preventing the locking mechanism from engaging the cassette or cartridge. The computer 104 may also provide a visual or auditory signal to the operator that the cartridge or cassette is not appropriate for use with the apparatus, prompting the operator to replace the cartridge or cassette.

While specific embodiments of the invention have been illustrated and described, it will be apparent that various modifications can be made without departing from the spirit and scope of the invention. Accordingly, it is intended that the invention not be limited, except as by the appended claims.

What is claimed is:

1. A method comprising:
a) sensing an identifier on a tubing cartridge or a pump/sensor cartridge mounted on an instrument;
b) determining if the cartridge is capable of being used with the instrument based on the identifier; and
c) locking the cartridge in place if the cartridge is capable of being used with the instrument.

2. The method of claim 1 further comprising emitting a warning if the cartridge is not capable of being used with the instrument.

3. The method of claim 1 wherein the identifier is a resistor, an RFID, or a bar code.

4. The method of claim 1 wherein the identifier further indicates whether the cartridge has been used.

5. The method of claim 1 wherein the identifier is located within a memory device associated with the cartridge.

6. The method of claim 1 wherein the instrument comprises a phacoemulsification instrument.
